(19) United States
(12) Patent Application Publication
(10) Pub. No.: US 2002/0168701 A1
(43) Pub. Date: Nov. 14, 2002

(54) Iron uptake and respiratory function are differentially regulated by yeast a kinases

(76) Inventors: Laura S. Robertson, Sheperdstown, WV (US); Helen Claire Causton, London (GB); Gerald R. Fink, Chestnut Hill, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 09/729,915

(22) Filed: Dec. 4, 2000

Related U.S. Application Data
(60) Provisional application No. 60/168,563, filed on Dec. 2, 1999.

(51) Int. Cl. C12Q 1/34

(52) U.S. Cl. 435/18

(57) ABSTRACT

Genes regulated by protein kinase A comprising the catalytic subunits encoded by Tpk1, Tpk2 or Tpk3 are described. Methods for altering iron uptake, trehalose breakdown, water homeostasis and respiratory growth as well as methods for altering branched chain amino acid synthesis are described. Further, methods for inhibiting virulence in an organism are described.

### RELATED APPLICATION

This application claims the benefit of U.S. Provisional Application No. 60/168,563 filed Dec. 2, 1999, the entire teachings of which are incorporated herein by reference.

### GOVERNMENT SUPPORT

The invention was supported, in whole or in part, by grant Numbers GM40266, GM34365 and GM35010 from the National Institutes of Health. The Government has certain rights in the invention.

### BACKGROUND OF THE INVENTION

Cyclic adenosine monophosphate (cAMP) is a naturally occurring compound that is present in all cells and tissues in organisms from bacteria to humans. In animal cells, cAMP appears to promote the expression of differentiated (specialized) properties and works as a second messenger. The cAMP signal transduction pathway controls a wide variety of processes. Most effects of cAMP in eukaryotic cells are mediated by activation of a single protein kinase, protein kinase A (PKA). PKA consists of two kinds of subunits, regulatory and catalytic. In yeast there exist three isoforms of PKA. In the presence of cAMP, the catalytic subunits are released and are enzymatically active. A conserved catalytic core exists in the structure that is shared by more than a hundred different protein kinases.

Yeast RAS proteins, which are structurally homologous to mammalian RAS oncoproteins, modulate adenylate cyclase, which in turn produces cAMP (Toda T. et al., Cell 50:277-287 (1987). The cAMP signal transduction pathway controls a wide variety of processes in fungi. In yeast, cAMP, acting through PKA, provides a key regulatory signal for growth on diverse carbon sources. Growth on fermentable carbon sources (glucose, fructose, sucrose; i.e., fermentive growth) requires a higher basal level of cAMP than does growth on nonfermentable carbon sources (ethanol, glycerol, acetate; i.e., respiratory growth). Therefore, the level of cAMP must decrease in order for cells to switch from growth on fermentable carbon sources to growth on non-fermentable carbon sources. This switch is known as the diauxic shift (Russell et al., Mol. Biol. Cell 4:757-765 (1993)). Addition of glucose to yeast cells growing on a non-fermentable carbon source or starved for glucose, results in a transient peak in intracellular cAMP levels. This transition to fermentation requires the transient increase of both cAMP and PKA (Jiang, Y. et al., EMBO J. 17:6942-6951)). Activated PKA shifts the metabolic flux away from gluconeogenesis and towards glycolysis by regulating key enzymes in these processes, including fructose-1,6-bisphosphatase and phosphofructokinase 2 (J. R. Broach and R. J. Deschenes, Adv. Cancer Res. 54:79-139 (1990)). Phosphorylation by PKA inactivates the transcription factor Adr1, a positive regulatory factor for the transcription of the respiratory enzyme Adh2 (Cherry J. R. et al., Cell 56:409-419 (1989)). In addition, PKA promotes the breakdown of glycogen and trehalose by inhibiting enzymes involved in synthesis (trehalose synthase and glycogen synthase) and activating enzymes involved in breakdown (trehalase and glycogen phosphorylase) of these storage carbohydrates.

Typically as glucose is depleted, transcription of genes involved in respiration, the TCA cycle, the glyoxylate cycle, gluconeogenesis, and storage carbohydrate synthesis is induced, whereas transcription of genes involved in glycolysis and protein synthesis is repressed (De Risi, J. L. et al., Science 278:680-686 (1997)). Consistent with the shift to respiratory growth, cytoplasmic ribosomal protein genes are repressed and mitochondrial ribosomal genes are induced. In view of the role of cAMP and the A kinases in the utilization of carbon sources, elucidation of the functions associated with the A kinases, particularly the catalytic subunits, in respiratory growth and their redundancy for these functions is needed. Further, in pathogenic fungi, regulation of these catalytic subunits provides tools for abrogating virulence and controlling the transition from bud-like growth to filamentous pseudohyphal growth required for pathogenicity.

### SUMMARY OF THE INVENTION

Yeast has three A kinase catalytic subunits which have greater than 75% identity and are encoded by the TPK genes (TPK1, TPK2 and TPK3) (Toda, T. et al. Cell 50: 277-87 (1987)). Although they are redundant for viability, the three A kinases are not redundant for pseudohyphal growth (Robertson, L. S. and Fink, G. R. Proc. Natl. Acad. Sci. USA 95:13783-7 (1998); Pan, X and Heitman, J. Mol Cell. Biol. 19: 4874-87 (1999)); Tpk2, but not Tpk1 or Tpk3, is required for pseudohyphal growth. As described herein, genome-wide transcriptional profiling has revealed unique gene expression signatures for each of the three A kinases leading to the identification of additional functional diversity among these proteins. Tpk2 negatively regulates genes involved in iron uptake and positively regulates genes involved in trehalose degradation and water homeostasis (Table 1). Tpk1 is required for the derepression of branched chain amino acid biosynthesis genes that have a second role in the maintenance of iron levels and DNA stability within mitochondria (Table 2). The fact that TPK2 deletion mutants grow better than wild type on non-fermentable carbon sources and on media deficient in iron supports the unique role of Tpk2 in respiratory growth and carbon source utilization.

Described herein is a method of altering iron uptake, trehalose breakdown, water homeostasis, respiratory growth or combinations thereof in a cell, comprising enhancing activity of protein kinase A in cell, whereby the expression of one or more genes responsive to protein kinase A which mediate iron uptake, trehalose breakdown, water homeostasis, respiratory growth or combinations thereof is altered, thereby altering iron uptake, trehalose breakdown, water homeostasis, respiratory growth or combinations thereof in cell.

In another embodiment, a methods of altering iron uptake in a cell, comprising altering activity of the protein kinase A catalytic subunit encoded by TPK2, whereby expression of one or more genes responsive to TPK2 which mediate iron uptake is altered wherein the activity of the protein kinase A catalytic subunit encoded by TPK2 is enhanced, thereby inhibiting iron uptake in the cell, wherein the cell is a fungal cell or a yeast cell wherein the activity of the protein kinase A catalytic subunit encoded by TPK2 is altered by altering the transcription of the TPK2 gene or altered by altering the expression of the TPK2 protein wherein the genes responsive to TPK2 are selected from the group consisting of FRE2, FRE3, FTR1, CCC2, SIT1, ARN1, YOL158c, YH1047c and combinations thereof are described.

In still another embodiment, methods of altering respiratory growth of a cell, comprising altering activity of the protein kinase A catalytic subunit encoded by TPK2, whereby expression of one or more genes responsive to TPK2 which mediate respiratory growth is altered are described whereby the activity of the protein kinase A catalytic subunit encoded by TPK2 is enhanced, thereby inhibiting respiratory growth of the cell wherein the cell is a fungal cell or a yeast cell; wherein the activity of the protein kinase A catalytic subunit encoded by TPK2 is altered by altering the transcription of the TPK2 gene or by altering the expression of the TPK2 protein; wherein the genes responsive to TPK2 are selected from the group consisting of FRE2, FRE3, FTR1, CCC2, SIT1, ARN1, YOL158c, YH1047c and combinations thereof are described.

In yet another embodiment, methods of altering trehalose degradation in a cell, comprising altering activity of the protein kinase A catalytic subunit encoded by TPK2, whereby expression of one or more genes responsive to TPK2 which mediate trehalose degradation is altered; wherein the activity of the protein kinase A catalytic subunit encoded by TPK2 is enhanced, thereby enhancing trehalose degradation in the cell; wherein the cell is a fungal cell or a yeast cell; wherein activity of the protein kinase A catalytic subunit encoded by TPK2 is altered by altering the transcription of the TPK2 gene or by altering the expression of the TPK2 protein; wherein the gene responsive to TPK2 is NTH1 are described.

In yet another embodiment, a method of altering trehalose degradation in a cell, comprising altering activity of the protein kinase A catalytic subunit encoded by TPK2, whereby expression of one or more genes responsive to TPK2 which mediate trehalose degradation is altered are described, wherein glycogen degradation is additionally altered in the cell is described.

In still another embodiment, methods of altering water homeostasis in a cell, comprising altering activity of the protein kinase A catalytic subunit encoded by TPK2, whereby expression of one or more genes responsive to TPK2 which mediate water homeostasis is altered; wherein the cell is a fungal cell or a yeast cell; wherein the activity of the protein kinase A catalytic subunit encoded by TPK2 is altered by altering the transcription of the TPK2 gene or by altering the expression of the TPK2 protein; wherein the gene responsive to TPK2 for altering water homeostasis in a cell is an aquaporin gene; wherein the aquaporin gene is AQY2 are described.

In yet another embodiment, methods of altering branched chain amino acid biosynthesis in a cell, comprising altering activity of the protein kinase A catalytic subunit encoded by TPK1, whereby expression of one or more genes responsive to TPK1 which mediate branched chain amino acid synthesis is altered; wherein the activity of the protein kinase A catalytic subunit encoded by TPK1 is enhanced, thereby enhancing branched chain amino acid synthesis in the cell; wherein the cell is a fungal cell or a yeast cell; wherein the activity of the protein kinase A catalytic subunit encoded by TPK1 is altered by altering the transcription of the TPK1 gene or altered by altering the expression of the TPK1 protein; wherein the genes responsive to TPK1 for altering branched chain amino acid biosynthesis are selected from the group consisting of BAT1, ILV5 and combinations thereof; wherein the genes responsive to TPK1 also have a role in the maintenance of iron levels and DNA stability within mitochondria are described.

In another embodiment, methods of inhibiting the transcription of a gene which mediates iron uptake in a cell, comprising enhancing activity of the protein kinase A catalytic subunit encoded by TPK2; wherein the genes responsive to TPK2 are selected from the group consisting of FRE2, FRE3, FTR1, CCC2, SIT1, ARN1, YOL158c, YH1047c and combinations thereof are described.

In yet another embodiment, methods of inhibiting the transcription of a gene which mediates respiratory growth in a cell, comprising enhancing activity of the protein kinase A catalytic subunit encoded by TPK2; wherein the genes responsive to TPK2 are selected from the group consisting of FRE2, FRE3, FTR1, CCC2, SIT1, ARN1, YOL158c, YH1047c and combinations thereof are described.

In still another embodiment, methods of enhancing the transcription of a gene which mediates trehalose degradation in a cell, comprising enhancing activity of the protein kinase A catalytic subunit encoded by TPK2; wherein the gene responsive to TPK2 is NTH1 are described.

In yet another embodiment, methods of enhancing the transcription of a gene which mediates water homeostasis in a cell, comprising enhancing activity of the protein kinase A catalytic subunit encoded by TPK2 are described. The gene responsive to TPK2 for mediating water homeostasis is AQY2 are described.

In still another embodiment, methods of enhancing the transcription of a gene which mediates branched chain amino acid synthesis in a cell, comprising enhancing activity of the protein kinase A catalytic subunit encoded by TPK1, whereby transcription of one or more genes responsive to TPK1 which mediate branched chain amino acid synthesis is altered, wherein the genes responsive to TPK2 are selected from the group consisting of BAT1, ILV5 and combinations thereof are described.

In another embodiment, methods of inhibiting virulence of an organism comprising enhancing activity of protein kinase A in one or more cells of said organism, whereby the expression of one or more genes responsive to protein kinase A which mediate iron uptake is inhibited, thereby inhibiting virulence of the organism are described wherein the genes responsive to protein kinase A for inhibiting virulence are selected from the group consisting of FRE2, FRE3, FTR1, CCC2, SIT1, ARN1, YOL158c, YH1047c and combinations thereof, wherein the organism is a fungus. A method of inhibiting virulence of an organism comprising enhancing activity of protein kinase A in one or more cells of said organism, whereby the expression of one or more genes responsive to protein kinase A which mediate iron uptake is inhibited, wherein capsule formation is prevented in the fungus is also described.

In yet another embodiment, methods of inhibiting virulence of an organism comprising enhancing activity of the protein kinase A catalytic subunit encoded by TPK2 in one or more cells of said organism, whereby the expression of one or more genes responsive to TPK2 which mediate iron uptake is inhibited, thereby inhibiting virulence of the organism, wherein the genes responsive to protein kinase A are selected from the group consisting of FRE2, FRE3, FTR1, CCC2, SIT1, ARN1, YOL158c, YH1047c and combinations thereof, wherein the organism is a fungus, wherein capsule formation is prevented in the fungus are described.

### BRIEF DESCRIPTION OF THE DRAWINGS

The FIGURE is a schematic of the process of high affinity iron uptake in yeast. Insoluble, extracellular Fe(III) is reduced to Fe(II) by the plasma membrane ferric reductases Fre 1 and Fre2. Iron is then transproted into the cell by a plasma membrane complex consisting of the multicopper oxidase Fet3 and the iron permease Ftr1. Ftr1 transports Fe(III); Fet3 oxidizes Fe(II) to Fe(III) to allow transport by Ftr1. Fet3 requires copper for activity. The copper transporter Ccc2 is required for the copper-loading of Fet3 in the late Golgi.

### DETAILED DESCRIPTION OF THE INVENTION

A description of preferred embodiments of the invention follows.

Expression arrays coupled with mutational analyses were used to elucidate differences between the three yeast PKA catalytic subunits encoded by the yeast TPK genes TPK1, TPK2 and TPK3 (Toda, T. et al., Cell 5:277-287 (1987)). The results of this analysis emphasizes that Tpk1, Tpk2 and Tpk3 are not functionally redundant, despite a high level of sequence identity at the amino acid level and overlapping roles in viability and many other functions. Use of expression arrays allowed the identification of those genes that are differentially regulated by the three catalytic subunits. Tpk2 specifically regulates genes involved in iron uptake, trehalose breakdown, and water homeostasis; Tpk1 specifically regulates a distinct set of genes with a putative role in respiration.

Expression of the high affinity iron uptake pathway genes (FRE2, FET3, FTR1 and CCC2), as well as expression of a family of genes related to the siderophore uptake gene SIT 1, were increased in TPK2 mutants. This is consistent with the growth phenotypes identified herein; TPK2 mutants grow better than wild type on ethanol/glycerol medium, and this differential growth is further enhanced by the addition of ferrozine. This is also consistent with the finding that strains whose only active A kinase is Tpk2 (in tpk1 TPK2 tpk3 bcyl strains) are defective for growth on acetate (as compared to wild type or to strains whose only active A kinase is Tpk1 or Tpk3) (Toda T. et al., Cell 50:277-287 (1987)). Thus Tpk2 inhibits respiratory growth through the negative regulation of iron uptake.

Data obtained from the analyses described herein supports the following paradigm that connects growth phases and iron metabolism in fungi. During fermentative growth on glucose, Tpk2, activated by cAMP, represses genes involved in iron metabolism. As glucose is depleted, Tpk2 activity is decreased, thereby relieving the repression of the iron transport systems. Derepression results in transport of iron into the cell where it is incorporated into respiratory enzymes that permit growth on non-fermentable carbon sources accumulating in the medium as the cells transition into the diauxic shift.

Iron transport must be carefully regulated because excess intracellular iron results in the generation of hydroxyl radicals that are toxic. This paradigm supports previous work indicating that the iron-regulated transcription factor Aft1 regulates high affinity iron uptake. FRE2, FET3, FTR1, and CCC2 are transcriptionally activated by Aft1 under conditions of iron deprivation, and FRE2 mRNA is undetectable in an aft1 mutant (Casas, C., Yeast, 13:621-637 (1997)). FRE2, FET3, FTR1, and CCC2, which are under Tpk2 control, contain Aft1 consensus binding sites in their promoter regions, and Aft1 has been shown to bind these sites (Yamaguchi-Iwai, Y. et al., EMBO J. 15:3377-3384 (1996)). aft1 null mutants grow on fermentable carbon sources, but not on nonfermentable carbon sources. This inability to grow on nonfermentable carbon sources is suppressed by the addition of ferrous iron to the growth medium, indicating that the aft1 growth defect is due to poor iron uptake (Casas, C., Yeast, 13:621-637 (1997)). These previous observation are consistent with a model in which Tpk2 negatively regulates Aft1 activity, which is required for expression of these high affinity iron uptake genes after the switch to respiratory growth.

As a result of the work described herein, targets of the cAMP pathways in fungi (e.g., yeast), and particularly genes that show strong regulation by the PKA catalytic subunits Tpk2 and Tpk1, have been identified (collectively “target genes”). These genes and their interaction with or regulation by Tpk2 can be targeted in methods of modulating (inhibiting or enhancing) the genes responsible for iron uptake, water homeostasis and trehalose breakdown, as well as methods of modulating the phenotypic effect mediated by these genes. These genes and their interaction with or regulation by Tpk1 can be targeted in a method of modulating (inhibiting or enhancing) the genes responsible for branched chain amino acid biosynthesis and the maintenance or mitochondrial iron levels and DNA stability, as well as methods of modulating the phenotypic effect mediated by these genes.

This model system can be applied to other species in which share the homology with the catalytic subunits of PKA. As used herein, PKA and its catalytic subunits Tpk1, Tpk2 and Tpk3 refer to the specific yeast genes described herein and structurally in Toda T. et al.,Cell 5:277-287 (1987) as well as homologs or varients of Tpk1, Tpk 2 and Tpk3 from other species. Homology includes, but is not limited to, the sequence similarity between two polypeptide molecules or two nucleic acid molecules. Two amino acid sequences are substantially homologous or substantially similar when greater than about 30% of the amino acids are identical, or greater than about 60% are similar (functionally identical). Preferably, the similar or homologous sequences are identified by alignment. Variants of the nucleic acid molecules encoding Tpk1, Tpk 2 or Tpk3 can be naturally occurring, such as allelic variants (same locus), homologs (different locus), and orthologs (different organism), or may be constructed by recombinant DNA methods or by chemical synthesis. Such non-naturally occurring variants can be made using well-known mutagenesis techniques, including those applied to polynucleotides, cells, or organisms. Accordingly, variants can contain nucleotide substitutions, deletions, inversions and/or insertions in either or both the coding and non-coding region of the nucleic acid molecule. Further, the variations can produce both conservative and non-conservative amino acid substitutions.

Typically, variants have a substantial identity with a nucleic acid molecule encoding Tpk1, Tpk 2 or Tpk 3 and the complements thereof. Particularly preferred are nucleic acid molecules and fragments which have at least about 60%, preferably at least about 70, 80 or 85%, more preferably at least about 90%, even more preferably at least about 95%, and most preferably at least about 98% identity with nucleic acid molecules described herein.

Such nucleic acid molecules can be readily identified as being able to hybridize under stringent conditions to a nucleotide sequence of Tpk1, Tpk 2 or Tpk 3 and the complements thereof. In one embodiment, the variants hybridize under high stringency hybridization conditions (e.g., for selective hybridization) to a nucleotide sequence of Tpk1, Tpk 2 and Tpk 3.

Stringent hybridization conditions for nucleic acid molecules are well known to those skilled in the art and can be found in standard texts such as Current Protocols in Molecular Biology, John Wiley & Sons, N.Y. (1998), pp. 2.10.1-2.10.16 and 6.3.1-6.3.6, the teachings of which are hereby incorporated by reference. As understood by those of ordinary skill, the exact conditions can be determined empirically and depend on ionic strength, temperature and the concentration of destabilizing agents such as formamide or denaturing agents such as SDS. Other factors considered in determining the desired hybridization conditions include the length of the nucleic acid sequences, base composition, percent mismatch between the hybridizing sequences and the frequency of occurrence of subsets of the sequences within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, preferably at least 40%, more preferably at least 60%, and even more preferably at least 70%, 80% or 90% of the length of the reference sequence. The actual comparison of the two sequences can be accomplished by well-known methods, for example, using a mathematical algorithm. A non-limiting example of such a mathematical algorithm is described in Karlin et al., Proc. Natl. Acad. Sci. USA, 90:5873-5877 (1993). Such an algorithm is incorporated into the NBLAST and XBLAST programs (version 2.0) as described in Altschul et al., Nucleic Acids Res., 25:389-3402 (1997). When utilizing BLAST and Gapped BLAST programs, the default parameters of the respective programs (e.g., NBLAST) can be used. See http://www.ncbi.nlm.nih.gov. In one embodiment, parameters for sequence comparison can be set at score=100, wordlength=12, or can be varied (e.g., W=5 or W=20).

A mathematical algorithm utilized for the comparison of sequences is the algorithm of Myers and Miller, CABIOS (1989). Such an algorithm is incorporated into the ALIGN program (version 2.0) which is part of the CGC sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci., 10:3-5; and FASTA described in Pearson and Lipman (1988) PNAS, 85:2444-8.

The percent identity between two amino acid sequences can be accomplished using the GAP program in the CGC software package (available at http://www.cgc.com) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the CGC software package (available at http://www.cgc.com), using a gap weight of 50 and a length weight of 3. Thus, a substantially homologous amino acid or nucleotide sequence means an amino acid or nucleotide sequence that is largely but not wholly homologous to Tpk1, Tpk2 or Tpk3, and which retains the same functional activity as the molecule to which it is homologous.

The invention relates to methods of altering iron uptake, trehalose breakdown, water homeostasis, respiratory growth, branched chain amino acid synthesis or combinations thereof in a cell by altering activity of protein kinase A in the cell, whereby the expression of one or more genes responsive to protein kinase A which mediate iron uptake, trehalose breakdown, water homeostasis, respiratory growth, branched chain amino acid synthesis or combinations thereof, respectively, is altered, thereby altering iron uptake, trehalose breakdown, water homeostasis, respiratory growth, branched chain amino acid synthesis or combinations thereof, respectively. That is, enhancement of PKA activity will enhance the expression of genes which are positively regulated by PKA, and inhibit the expression of genes which are negatively regulated by PKA. Conversely, inhibition of PKA activity will inhibit the expression of genes which are positively regulated by PKA and enhance the expression of genes which are negatively regulated by PKA. Genes which are positively or negatively regulated by PKA isoforms which comprise the TPK1 or TPK2 catalytic subunit are described herein in detail. For example, genes which mediate the iron uptake pathway are disclosed herein to be negatively regulated by TPK2, and correspondingly by the PKA isoform which comprises the TPK2 catalytic subunit. The PKA-responsive genes which regulate the other phenotypic pathways described herein are also described.

Alteration of PKA activity can occur in a number of ways which will be readily recognized by the skilled artisan. For example, transcription of the genes encoding the regulatory and/or catalytic subunits of PKA can be increased. Alternatively, gene therapy methods can be used to introduce exogenous PKA subunit-encoding nucleic acid molecules into a cell to increase the amount of PKA produced in the cell. Other suitable methods will be apparent to the skilled artisan. Alteration of PKA activity is intended to encompass any quantitative or qualitative difference in the amount, duration, efficiency or potency of PKA enzymatic activity.

Also described are methods of altering iron uptake in a cell, by altering activity of the protein kinase A catalytic subunit encoded by TPK2, thus altering expression of one or more genes responsive to TPK2 which mediate iron uptake. The activity of the protein encoded by the TPK2 gene can be enhanced or inhibited. The activity of the protein kinase A catalytic subunit encoded by TPK2 can be altered by altering the transcription of the TPK2 gene or altered by the expression of the TPK2 protein. The activity can be altered quantitatively or qualitatively. The activity can be an increase in protein activity where the protein is altered so that the same amount of protein produces longer or stronger effects of the protein. The cell can be a fungal or yeast cell. The TPK2 responsive genes for altering iron uptake are FRE2, FRE3, FTR1, CCC2, SIT1, ARN1, YOL158c, YH1047c and combinations of the genes.

In another embodiment, methods of altering respiratory growth of a cell by altering activity of the protein kinase A catalytic subunit encoded by TPK2, whereby expression of one or more genes responsive to TPK2 which mediate respiratory growth is altered are described. The activity of the protein kinase A catalytic subunit encoded by TPK2 can be enhanced or inhibited and can be altered by altering transcription of the TPK2 gene or expression of the TPK2 protein. The cell can be a fungal cell or a yeast cell. The TPK2 responsive genes for altering respiratory growth are FRE2, FRE3, FTR1, CCC2, SIT1, ARN1, YOL158c, YH1047c and combinations of the genes.

In still another embodiment, methods of altering trehalose degradation is described by altering activity of the protein kinase A catalytic subunit encoded by TPK2, whereby expression of one or more genes responsive to TPK2 which mediate respiratory growth is altered are described. The activity of the protein kinase A catalytic subunit encoded by TPK2 can be enhanced or inhibited and can be altered by altering transcription of the TPK2 gene or expression of the TPK2 protein. The cell can be a fungal cell or a yeast cell. The TPK2 responsive gene for altering trehalose degradation is NTH1. Additionally, glycogen degradation is altered in a cell.

In another embodiment, methods of altering water homeostasis in a cell by altering activity of the protein kinase A catalytic subunit encoded by TPK2, whereby expression of one or more genes responsive to TPK2 which mediate respiratory growth is altered are described. The activity of the protein kinase A catalytic subunit encoded by TPK2 can be enhanced or inhibited and can be altered by altering transcription of the TPK2 gene or expression of the TPK2 protein. The cell can be a fungal cell or a yeast cell. The TPK2 responsive gene which alters water homeostasis is an aquaprotin gene, specifically AQY2.

Methods are also described for altering branched chain amino acid biosynthesis in a cell, by altering activity of the protein kinase A catalytic subunit encoded by TPK1, thus altering expression of one or more genes responsive to TPK1 which mediate branched chain amino acid synthesis. The activity of the protein kinase A catalytic subunit encoded by TPK1 can be enhanced or inhibited and can be altered by altering transcription of the TPK1 gene or expression of the TPK 1 protein. The cell can be a fungal cell or a yeast cell. The TPK1 responsive genes which mediate branched chain amino acid synthesis are BAT1, ILV5 and combinations. These genes responsive to TPK1 also have a role in the maintenance of iron levels with DNA stability within mitochondria.

Methods are also described for inhibiting the transcription of a gene which mediates iron uptake or respiratory growth or trehalose degradation or water homeostasis by enhancing activity of the protein kinase A catalytic subunit encoded by TPK2.

In yet another embodiment, methods are described of inhibiting virulence of an organism (i.e. fungus) by enhancing activity of protein kinase A, or specifically TPK2 in one or more cells in the organism thus inhibiting expression of one or more genes responsive to protein kinase A or one or more genes responsive to TPK2, which mediate iron uptake. Iron limitation is required for capsule formation which is required for virulence. Dimorphism, is required for virulence in some species. Tpk2 is essential for pseudohyphal growth in yeast and thus is essential for organisms to which from a budlike form (fermentive growth) to pathogenic filamentous form (respiratory growth). cAMP levels have also been connected to virulence traits in the human pathogen Cryptococcus neoformans (Kronstad J., et al., Arch. Microbiol 170(6):395-404(1998)).

Compounds or molecules with modulate the target genes identified herein, directly or through their regulation by Tpk1 or Tpk2, can be identified by means, for example, of an assay in which one or more of the genes (e.g., a gene encoding a protein involved in the high affinity iron uptake pathway) is expressed in an appropriate host cell and the effects of a candidate modulator shown to decrease expression are inhibitors of a gene shown, as described herein to be regulated by Tpk1 or Tpk2); candidate modulators shown to increase expression are enhancers of such a Tpk-regulated genes. In addition, fungal genes responsible for iron uptake regulated by Tpk2 can be targeted to modulate fungal host interaction. These genes can be targeted, for example, to inhibit fungal invasion by increasing iron uptake thus inhibiting capsule formation which requires iron limitation. Also, fungal genes responsible for pseudohyphal growth can be targeted to inhibit pathogens where dimorphism and the transition to pseudohyphal growth is required for virulence. Inhibition of Tpk2, directly or indirectly (e.g., by inhibiting a gene or the product of a gene with which Tpk2 interacts) will result in the inhibition of pseudohyphal growth. Inhibitors and enhancers of genes regulated by Tpk2 and Tpk1 and the genes in turn regulated by the Tpk2 or Tpk1-responsive pathways are encompassed by this invention. Compounds with enhance or activate the catalytic subunits Tpk2 or Tpk 1 are activators, and conversely those which repress or decrease expression or activity are inhibitors. An agonist of the catalytic subunit is a compound which exhibits a biological activity of the catalytic subunit. An antagonist of the catalytic subunit means a compound which blocks, inhibits or decreases the biological activity of the catalytic subunit.

Agents for use in the methods of the invention include nucleic acid molecules (e.g., antisense), polypeptides and proteins, antibodies and small organic molecules. Suitable formulations of agents for use in this invention can include, for example, powders, liquids, aerosols, gels and other formulations known to the skilled artisan. The present invention also pertains to pharmaceutical compositions comprising agents identified according to the invention for use in the treatment of fungal invasion. For instance, the agent identified according to the present invention can be formulated with a physiologically acceptable medium to prepare a pharmaceutical composition. The particular physiological medium may include, but is not limited to, water, buffered saline, polyols (e.g., glycerol, propylene glycol, liquid polyethylene glycol) and dextrose solutions. The optimum concentration of the active ingredient(s) in the chosen medium can be determined empirically, according to procedures well known to medicinal chemists, and will depend on the ultimate pharmaceutical formulation desired. In organisms other than plants, methods of administration of pharmaceutical compositions include, but are not limited to, intradermal, intramuscular, intraperitoneal, intravenous, subcutaneous, oral and intranasal. Other suitable methods of introduction can also include rechargeable or biodegradable devices and slow release polymeric devices. The pharmaceutical compositions of this invention can also be administered as part of a combinatorial therapy with other agents.

Also encompassed is a method of inhibiting (totally or partially) invasion of the host, particularly a plant host by a fungus. In the method, a compound or molecule which inhibits the pathways responsive to Tpk2 or specifically inhibits Tpk2 is applied to the host in such a manner that it contacts the fungus and inhibits one or more components of the Tpk2 responsive pathway or Tpk2 and inhibits the fungus' ability to invade.

In a further embodiment, methods are disclosed for identifying genes regulated by only one catalytic subunit of a kinase containing multiple subunits while eliminating non-specific kinase effects, by obtaining mutant strains deleted for one catalytic subunit; obtaining RNA from the strains; hybridizing cDNA to high density genomic arrays; and analyzing data. If expression changes in one mutant strain but remains constant among the other catalytic mutant strains and wild type, the resulting expression is indicative of the regulation activity of that catalytic subunit.

TABLE 1

| | |
|---|---|
| Protein | Function |

Tpk2 regulates the high-affinity iron-uptake pathway

| | |
|---|---|
| High-affinity iron uptake | |
| Fre1 | Ferric reductase, plasma membrane |
| Fre2 | Ferric reductase, plasma membrane |
| Fet3 | Multicopper ferroxidase, plasma |
| | membrane |
| Ftr1 | Iron permease, plasma membrane |
| Ccc2 | Copper transporter |
| Low-affinity iron uptake | |
| Fet4 | Low-affinity Fe(II) transporter |
| Vacuole iron transport | |
| Fth1 | Vacuolar iron transporter, FTR1 |
| | homolog |
| Fet5 | Multicopper oxidase, vacuole |
| Mitochondrial iron homeostasis | |
| Atm1 | ABC transporter, mitochondria |
| Yfh1 | Yeast frataxin homolog |
| Putative iron transporters | |
| Sit1 | Siderophore iron transport |
| Arn1 | MFS/MDR family |
| Yol158c | MFS/MDR family |
| Yhl047c | MFS/MDR family |

Tpk2 positively regulates trehalose breakdown

| | |
|---|---|
| Nth1 | trehalase |

Tpk2 positively regulates water homeostasis

| | |
|---|---|
| Agy2 | aquaporin |

Proteins involved in iron transport are listed above. Those shown in bold are negatively regulated by Tpk2. ABC, ATP-binding cassette; MFS/MDR, major facilitator super family/multidrug resistance.

TABLE 2
Tpk1 regulates genes of the branched amino acid pathway that have a
second function in mitochondrial iron homeostasis and
mitochondrial DNA stability

| Protein | Function |
|---|---|
| Bat1 | mitochondrial branched chain |
| | amino acid transamiase |
| Ilv5 | ketol-acid reductoisomerase |
| Yrb5 | nuclear pore protein |

These proteins are derepressed by Tpk1

### EXAMPLES

Materials and Methods

Yeast Strains

Genotypes of the strains used are 10560-2B MATa ura3-52 his3::hisG leu2::hisG. LRY765 MATa ura3-52 his3::hisG leu2::hisG tpk1::URA3, LRY520 MATa ura3-52 his3::hisG trp1::hisG tpk1::URA3, LRY590 MATa ura3-52 his3::hisG leu2::hisG tpk2::HIS3, and LRY636 MATa ura3-52 his3::hisG leu2::hisG tpk3::HIS3. All strains are congenic to the Σ1278b background.

Growth Media and Plate Phenotypes

Standard yeast genetic techniques and growth media were used (Guthrie, C. & Fink G. R., Guide to Yeast Genetics and Molecular Biology, Academic Press, Inc., San Diego, Calif. (1991)). For Northerns and transcriptional profiling, strains were grown in liquid YPD at 30° C. and 250 rpm. For plate phenotypes, ten-fold dilutions of the strains 10560-2B, LRY765, LRY590, and LRY636 were spotted onto an agar plate and grown at 30° C. YPD is yeast extract, peptone medium supplemented with 2% glucose. YPE/G is yeast extract, peptone medium supplemented with 2% ethanol and 2% glycerol. Low iron medium is synthetic minimal medium (SD) without ferric chloride and buffered to pH 7.0 using MES-Tris. The iron chelator ferrozine was spread on top of agar medium to a final concentration of 0.5 mM.

Genome-Wide Trascriptional Profiling 10560-2B, LRY520, LRY765, LRY590, and LRY636 were used in the array experiments. Duplicate cultures for each strain were grown and processed separately. Yeast cultures grown in YPD were harvested during early to mid exponential phase. Total RNA was extracted, and polyadenylated RNA was selected from each sample. Target cDNA was produced, cDNA was hybridized to high-density oligonucleotide arrays, and the arrays were stained, washed and scanned using the methods of D. J. Lockhart et al., Nat. Biotechnol.14:1675-80 (1996) and L. Wodicka et al., Nat. Biotechnol. 15:1359-67 (1997). Expression measurements and scaling were done according to Galitski et al, Science 285:251-254 (1999). The data was analyzed using the web-based tool GEAP developed by Tim Galitski and Alok Saldanha (Whitehead Institute, Cambridge, Mass.). For the primary data analysis, a quantitative variation filter was applied requiring that the data from at least one strain showed an average difference greater than 100 for a gene to be considered in the data set. This filtered data set was then examined using nearest neighbor analysis with a Euclidean distance metric to find genes whose expression differed in only one mutant strain compared to both wild type and the other two mutant strains. Ten random data sets (within-gene permutations of the experimental data set) were analyzed using the same criteria to determine the distance for each input query that generated on average less than one false positive. This stringent requirement results in high specificity, but low sensitivity. Two less stringent standards were also used for secondary analysis: (a) genes whose expression changed at least two fold in a mutant strain compared to wild type and whose expression was consistent in duplicate experiments; and (b) genes from the unfiltered data set that are nearest neighbors using a Euclidean distance metric of genes found in the preliminary analysis.

Northern Analysis

10560-2B, LRY765, LRY590, and LRY636 were used in the Northern analysis. Strains were grown in liquid YPD at 30° C. to an OD₆₀₀ of approximately 1.0. Total RNA was harvested and 10 μg RNA was loaded per lane (Ausubel, F.M., et al., in Current Protocols in Molecular Biology, ed. Chanda, V.B, Wiley, New York, (1991)). Northerns were probed with FET3, FTR1, FRE1, and SIT1. The entire open reading frames of these genes were PCR amplified from genomic DNA using GENEPAIRS™ obtained from Research Genetics (Alabama USA). The exposure time for FET3 was 45 minutes at−80° C., for FRE1 and FTR1 15.5 hours at −80° C., and for SIT1 12 days at room temperature.

Respiratory Growth Phenotypes of TPK Mutants

Wild type, TPK1, TPK2, and TPK3 mutant strains were grown on ethanol/glycerol medium in the presence and absence of the iron chelator, ferrozine, to test for phenotypic consequences of the transcriptional effects seen in the array experiments. Since iron is required as a cofactor for several respiratory enzymes, TPK2 mutants, which over express these iron uptake genes, might show enhanced growth on medium that forces yeast to respire. Reciprocally, TPK1 mutants can have a growth defect on medium that forces yeast to respire, because Tpk1 is required for derepression of BAT1 and ILV5, genes that can regulate respiratory function through their role in maintenance of mitochondrial iron levels and mitochondrial DNA.

Results

### Example 1

Differentially-Regulated Genes

To identify genes regulated specifically by only one of the PKA catalytic subunits, deletion mutants lacking only one of the subunits, but expressing the other two, were used. Target cDNA from these strains were hybridized to high density genomic arrays. Data analysis identified those genes whose expression had changed in one of the mutant strains, but whose expression remained constant in the other data sets. This approach eliminated non-specific PKA effects such as functions required for viability and functions mediated redundantly by the A kinases.

For each of the three mutant strains (tpk1, tpk2, and tpk3), the percentage of the genome affected was relatively small; expression increased at least two-fold compared to wild type for approximately 4% of the genome and decreased at least two-fold for approximately 4%. This effect is similar to that of some transcription factors such as Gcn5, Swi2, or Srb10, but less than the overall change in transcription associated with the diauxic shift. Deletion of one catalytic subunit does not lead to increased expression of the other two subunits, showing that cells do not compensate for the loss of one catalytic subunit by over expression of another. For each of the mutants, approximately 50 different genes were identified which showed a unique, tpk-specific expression profile.

Array data obtained as described herein confirmed several observations made previously. In particular, the FLO11 expression pattern obtained from arrays described herein was very similar to that previously determined by Northern blot analysis (Robertson, L. S., et al., Proc. Nat. Acad. Sci. USA 95:13783-13787 (1998)); that is, FLO11 expression was essentially unchanged in a TPK1 mutant (0.8 times wild type), drastically reduced in a TPK2 mutant (0.05×wt), and increased in a TPK3 mutant (2.6 times wild type). Another cell surface flocculin gene, FLO10, showed a very similar expression pattern: 1.3-fold increase in tpk1 strains, an approximately three fold decrease in tpk2 strains (0.3 times wild type) and a 12.7-fold increase in tpk3 strains. The expression of other known flocculin genes, FLO1, FLO5, FLO8, and FLO9, was essentially unchanged in the TPK mutants.

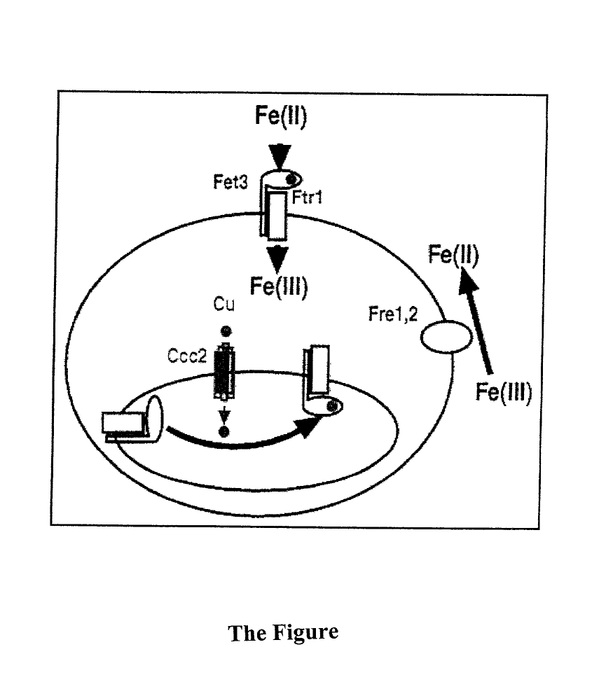

Tpk2 function is required for transcriptional repression of the entire high affinity iron uptake pathway (FIG. 1 and Table 1). The array data show that expression of FTR1 and FRE2 is more than two-fold higher and that expression of FET3 and CCC2 was also higher in a TPK2 mutant. Fre1 and Fre2 are plasma membrane ferric reductases (Danceis, A. et al., Proc. Nat. Acad. Sci. USA 89:3869-3873 (1992) and Georgatsou, E. et al., Mol. Cell. Biol. 14:3065-3073 (1994)) that reduce insoluble, extracellular iron Fe(III) to soluble Fe(II). The soluble iron is transported into the cell via a high affinity system consisting of the plasma membrane complex of the multicopper oxidase Fet3 (the yeast homolog of ceruloplasmin) and the transporter Ftr1. Ftr1 transports the oxidized form Fe(III); this reoxidation of Fe(II) to Fe(III) is catalyzed by Fet3 (Askwith, C. et al., Cell 76:403-410 (1994); Stearman, R. et al., Science 271:1552-1557(1996)). The P-type ATPase Ccc2 (the yeast homolog of Menkes-Wilson protein) is required for loading copper onto Fet3 (Yuan, D. S., et al,. Proc. Nat. Acad. Sci. USA 92:2632-2636 (1995)).

The connection of Tpk2 to iron uptake was strengthened by the finding herein that expression of SIT1 (siderophore iron transport), a gene whose function is required for the uptake of the siderophore ferrioxamine B (Lesuisse E. et al., Microbiology 144: 3455-3462 (1998)), is also increased in tpk2 strains. SIT1 is one of four genes in the major facilitator super family/multidrug resistance (MFS/MDR) that have greater than two-fold increased expression in TPK2 mutants. This large family of putative permeases and transporters is predicted to include 186 yeast proteins; however, these four genes (YOL158c, SIT1, YHL047c, and ARN1) are closely related and form a distinct sub-group based solely on sequence (Nelissen et al., FEMS Microbiol. Rev. 21:113-134 (1997)). Since transcription of YOL158c, YHL047c, and ARN1 is regulated in a manner similar to that of the high affinity iron uptake pathway and SIT1, these three genes could also be involved in iron uptake. The regulation of iron uptake genes by Tpk2 was confirmed by Northern blotting.

Tpk1 is required for the derepression of both BAT1 and ILV5, as expression of BAT1 is reduced 2.4-fold and expression of ILV5 is reduced 1.4-fold in a tpk1 strain compared to wild type. Bat1 is the mitochondrial branched chain amino acid transaminase (Eden, A. et al., J Biol. Chem. 271: 20242-20245 (1996); Kispal G. et al., J Biol. Chem. 271: 24458-24464 (1996)). I1v5 is a keto-acid reductoisomerase that catalyzes an early step in the biosynthesis of valine, isoleucine, and leucine. In addition to its role in branched chain amino acid biosynthesis, BAT1 appears to be involved in exit from stationary phase. Upon exit from stationary phase there is a transient spike in cAMP, activation of the PKA's, and cellular reprogramming of transcription that mediates the return to growth. Cells that are deficient in one of the two BAT genes display no obvious growth reduction during exponential growth, but are slow to leave stationary phase in comparison with wild type cells (Kispal G. et al., J. Biol. Chem. 271: 24458-24464 (1996)).

Bat1 could also play a role in maintaining mitochondrial iron homeostasis and mitochondrial DNA stability via an interaction with Atm1. Atm1, an ABC transporter required for iron homeostasis, is located in the mitochondrial inner membrane (J. Leighton and G. Schatz, EMBO J. 14:188-195 (1995)). Cells lacking Atm1 accumulate very high levels of iron in their mitochondria and are unable to grow on nonfermentable carbon sources (Kispal. G. et al., FEBS Lett. 418:346-350 (1997)). Overexpression of BAT1 is believed to stabilize the temperature-sensitive Atm1 at the non-permissive temperature (Eden, A. et al., J Biol. Chem. 271: 20242-20245 (1996)). One model which explains this data is that in the absence of Tpk1, BAT1 expression is reduced and the level of iron in the mitochondrion rises. High levels of iron result in increased loss of mitochondrial DNA and consequently loss of mitochondrial function. Null mutations in ILV5 result in the ρ petite phenotype in which large segments of the mitochondrial genome are deleted. Over expression of ILV5, but not ILV2, another branched chain amino acid pathway gene, suppresses the mutant phenotype of abf2 strains (Zelenaya-Troiskaya, O. et al., EMBO J 14:3268-3276 (1995)). Abf2 is a DNA binding protein required for the maintenance of mtDNA on glucose (J. F. Diffley and B. Stillman, Proc. Nat. Acad. Sci. USA 88:7864-7868 (1991) and J. F. Diffley and B. Stillman, J. Biolo. Chem 267:3368-3374(1992)). abf2 null mutants are deficient in respiration (J. F. Diffley and B. Stillman, J. Biol. Chem. 267:3368-3374(1992)) Together these facts indicate that I1v5 is required for the stability of the mitochondrial genome and that Tpk1 regulates mitochondrial proteins key to this process.

### Example 2

Growth Phenotypes

Remarkably, TPK2 mutants grow better than wild type on ethanol/glycerol medium, and this difference is enhanced in the presence of ferrozine In contrast, TPK1 mutants have a growth defect on ethanol/glycerol, ethanol/glycerol containing ferrozine, and low iron/glucose media. These phenotypes support opposing functions for Tpk1 and Tpk2 in respiration. Expression of NTH1, whose product breaks down trehalose into its constituent glucose molecules, was reduced approximately three-fold in a TPK2 mutant. Trehalose is a storage carbohydrate also involved in resistance to stress. Activity of Nth1 decreases 95% at the diauxic shift, at the same time that Nth1 is dephosphorylated (Coutinho, C. Biochem. Int., 26:521-530 (1992)). Nth1 contains two consensus PKA phosphorylation sites (RRxS) and its activity in vitro is stimulated by cAMP (Nwaka, S. and Holzer, H. Prog. Nucleic Acid Res. Mol. Biol. 58:197-237 (1998)). In addition to this possible post-translational activation by PKA, transcription of Nth1 is also positively regulated by Tpk2.

Aquaprotin AQY2 expression was reduced approximately three-fold in a TPK2 mutant. Aquaporins are involved in the maintenance of water homeostasis in cells. There are four aquaporin family genes in yeast: two aquaglyceroproteins permeable to both water and glycerol, FPS1 and YFL054, and two orthodox aquaporins permeable only to water, AQY1 and AQY2. The two orthodox aquaporins Aqy1 and Aqy2 are both nonfunctional in the laboratory strain S288c, but are functional water channels in the strains profiled here (Σ1278b, Bonhivers, M. et al, J. Biol. Chem. 273:27565-27572 (1998); Laize, V., et al., Biochem. Biophys. Res. Commun. 257: 139-144 (1999)). In mammalian cells, aquaporins have been shown to be regulated both transcriptionally and post-translationally by PKA (Knepper M. A. and Inoue, T. Curr. Opin. Cell Biol. 9:560-564 (1997)). AQY2, but not AQY1, was shown to be transcriptionally regulated by the Tpk2 kinase. Although the subcellular localization of these aquaporins is not known, Aqy2 could be responsible for uptake of water into the vacuole. As the vacuole is an important organelle for iron and copper detoxification (Szczypka, M.S., et al., Yeast 13: 1423-1435 (1997)), it is reasonable to posit that the regulation of AQY2 by Tpk2 is another signature of the iron uptake pathway.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of altering iron uptake, trehalose breakdown, water homeostasis, respiratory growth or combinations thereof in a cell, comprising enhancing activity of protein kinase A in said cell, whereby the expression of one or more genes responsive to protein kinase A which mediate iron uptake, trehalose breakdown, water homeostasis, respiratory growth or combinations thereof is altered, thereby altering iron uptake, trehalose breakdown, water homeostasis, respiratory growth or combinations thereof in said cell.

2. A method of altering iron uptake in a cell, comprising altering activity of the protein kinase A catalytic subunit encoded by TPK2, whereby expression of one or more genes responsive to TPK2 which mediate iron uptake is altered, thereby altering iron uptake in the cell.

3. A method according to claim 2, wherein activity of the protein kinase A catalytic subunit encoded by TPK2 is enhanced, thereby inhibiting iron uptake in the cell.

4. A method according to claim 2, wherein the cell is a fungal cell.

5. A method according to claim 4, wherein the fungal cell is a yeast cell.

6. A method according to claim 2, wherein activity of the protein kinase A catalytic subunit encoded by TPK2 is altered by altering the transcription of the TPK2 gene.

7. A method according to claim 2, wherein activity of the protein kinase A catalytic subunit encoded by TPK2 is altered by altering the expression of the TPK2 protein.

8. A method according to claim 2, wherein the genes responsive to TPK2 are selected from the group consisting of FRE2, FRE3, FTR1, CCC2, SIT1, ARN1, YOL158c, YH1047c and combinations thereof.

9. A method of altering respiratory growth of a cell, comprising altering activity of the protein kinase A catalytic subunit encoded by TPK2, whereby expression of one or more genes responsive to TPK2 which mediate respiratory growth is altered, thereby altering respiratory growth of the cell.

10. A method according to claim 9, wherein activity of the protein kinase A catalytic subunit encoded by TPK2 is enhanced, thereby inhibiting respiratory growth of the cell.

11. A method according to claim 9, wherein the cell is a fungal cell.

12. A method according to claim 11, wherein the fungal cell is a yeast cell.

13. A method according to claim 9, wherein activity of the protein kinase A catalytic subunit encoded by TPK2 is altered by altering the transcription of the TPK2 gene.

14. A method according to claim 9, wherein activity of the protein kinase A catalytic subunit encoded by TPK2 is altered by altering the expression of the TPK2 protein.

15. A method according to claim 9, wherein the genes responsive to TPK2 are selected from the group consisting of FRE2, FRE3, FTR1, CCC2, SIT1, ARN1, YOL158c, YH1047c and combinations thereof.

16. A method of altering trehalose degradation in a cell, comprising altering activity of the protein kinase A catalytic subunit encoded by TPK2, whereby expression of one or more genes responsive to TPK2 which mediate trehalose degradation is altered, thereby altering trehalose degradation in the cell.

17. A method according to claim 16, wherein activity of the protein kinase A catalytic subunit encoded by TPK2 is enhanced, thereby enhancing trehalose degradation in the cell.

18. A method according to claim 16, wherein the cell is a fungal cell.

19. A method according to claim 18, wherein the fungal cell is a yeast cell.

20. A method according to claim 16, wherein activity of the protein kinase A catalytic subunit encoded by TPK2 is altered by altering the transcription of the TPK2 gene.

21. A method according to claim 16, wherein activity of the protein kinase A catalytic subunit encoded by TPK2 is altered by altering the expression of the TPK2 protein.

22. A method according to claim 16, wherein the gene responsive to TPK2 is NTH1.

23. A method according to claim 16, wherein glycogen degradation is additionally altered in the cell.

24. A method of altering water homeostasis in a cell, comprising altering activity of the protein kinase A catalytic subunit encoded by TPK2, whereby expression of one or more genes responsive to TPK2 which mediate water homeostasis is altered, thereby altering water homeostasis in the cell.

25. A method according to claim 24, wherein the cell is a fungal cell.

26. A method according to claim 24, wherein the fungal cell is a yeast cell.

27. A method according to claim 24, wherein activity of the protein kinase A catalytic subunit encoded by TPK2 is altered by altering the transcription of the TPK2 gene.

28. A method according to claim 24, wherein activity of the protein kinase A catalytic subunit encoded by TPK2 is altered by altering the expression of the TPK2 protein.

29. A method according to claim 24, wherein the gene responsive to TPK2 is an aquaporin gene.

30. A method according to claim 29, wherein the aquaporin gene is AQY2.

31. A method of altering branched chain amino acid biosynthesis in a cell, comprising altering activity of the protein kinase A catalytic subunit encoded by TPK1, whereby expression of one or more genes responsive to TPK1 which mediate branched chain amino acid synthesis is altered, thereby altering branched chain amino acid synthesis in the cell.

32. A method according to claim 31, wherein activity of the protein kinase A catalytic subunit encoded by TPK1 is enhanced, thereby enhancing branched chain amino acid synthesis in the cell.

33. A method according to claim 31, wherein the cell is a fungal cell.

34. A method according to claim 33, wherein the fungal cell is a yeast cell.

35. A method according to claim 31, wherein activity of the protein kinase A catalytic subunit encoded by TPK1 is altered by altering the transcription of the TPK1 gene.

36. A method according to claim 31, wherein activity of the protein kinase A catalytic subunit encoded by TPK1 is altered by altering the expression of the TPK1 protein.

37. A method according to claim 31, wherein the genes responsive to TPK1 are selected from the group consisting of BAT1, ILV5 and combinations thereof.

38. A method according claim 31, wherein the genes responsive to TPK1 also have a role in the maintenance of iron levels and DNA stability within mitochondria.

39. A method of inhibiting the transcription of a gene which mediates iron uptake in a cell, comprising enhancing activity of the protein kinase A catalytic subunit encoded by TPK2, whereby transcription of one or more genes responsive to TPK2 which mediate iron uptake is altered.

40. A method according to claim 39, wherein the genes responsive to TPK2 are selected from the group consisting of FRE2, FRE3, FTR1, CCC2, SIT1, ARN1, YOL158c, YH1047c and combinations thereof.

41. A method of inhibiting the transcription of a gene which mediates respiratory growth in a cell, comprising enhancing activity of the protein kinase A catalytic subunit encoded by TPK2, whereby transcription of one or more genes responsive to TPK2 which mediate respiratory growth is altered.

42. A method according to claim 41, wherein the genes responsive to TPK2 are selected from the group consisting of FRE2, FRE3, FTR1, CCC2, SIT1, ARN1, YOL158c, YH1047c and combinations thereof.

43. A method of enhancing the transcription of a gene which mediates trehalose degradation in a cell, comprising enhancing activity of the protein kinase A catalytic subunit encoded by TPK2, whereby transcription of one or more genes responsive to TPK2 which mediate trehalose degradation is altered.

44. A method according to claim 43, wherein the gene responsive to TPK2 is NTH1.

45. A method of enhancing the transcription of a gene which mediates water homeostasis in a cell, comprising enhancing activity of the protein kinase A catalytic subunit encoded by TPK2, whereby transcription of one or more genes responsive to TPK2 which mediate water homeostasis is altered.

46. A method according to claim 45, wherein the gene responsive to TPK2 is AQY2.

47. A method of enhancing the transcription of a gene which mediates branched chain amino acid synthesis in a cell, comprising enhancing activity of the protein kinase A catalytic subunit encoded by TPK1, whereby transcription of one or more genes responsive to TPK1 which mediate branched chain amino acid synthesis is altered.

48. A method according to claim 47, wherein the genes responsive to TPK2 are selected from the group consisting of BAT1, ILV5 and combinations thereof.

49. A method of inhibiting virulence of an organism comprising enhancing activity of protein kinase A in one or more cells of said organism, whereby the expression of one or more genes responsive to protein kinase A which mediate iron uptake is inhibited, thereby inhibiting virulence of the organism.

50. A method according to claim 49, wherein the genes responsive to protein kinase A are selected from the group consisting of FRE2, FRE3, FTR1, CCC2, SIT1, ARN1, YOL158c, YH1047c and combinations thereof.

51. A method according to claim 50, wherein the organism is a fungus.

52. A method according to claim 51, wherein capsule formation is prevented in the fungus.

53. A method of inhibiting virulence of an organism comprising enhancing activity of the protein kinase A catalytic subunit encoded by TPK2 in one or more cells of said organism, whereby the expression of one or more genes responsive to TPK2 which mediate iron uptake is inhibited, thereby inhibiting virulence of the organism.

54. A method according to claim 53, wherein the genes responsive to protein kinase A are selected from the group consisting of FRE2, FRE3, FTR1, CCC2, SIT1, ARN1, YOL158c, YH1047c and combinations thereof.

55. A method according to claim 53, wherein the organism is a fungus.

56. A method according to claim 55, wherein capsule formation is prevented in the fungus.
